(19) United States
(12) Plant Patent Application Publication
(10) Pub. No.: US 2005/0138711 P1
(43) Pub. Date: Jun. 23, 2005

(54) GOATSBEARD PLANT NAMED 'MISTY LACE'

(50) ARUNCUS DIOICUS X A. AETHUSIFOLIUS; MISTY LACE

(76) Inventors: Allan M. Armitage, Athens, GA (US); Hazel Y. Wetzstein, Athens, GA (US)

Correspondence Address: MACCORD MASON PLLC, 300 N. GREENE STREET, SUITE 1600, P. O. BOX 2974, GREENSBORO, NC, 27402, US

(21) Appl. No.: 10/738,072

(22) Filed: Dec. 17, 2003

(51) Int. Cl. A01H 5/00

(52) U.S. Cl. PLT/263

(57) ABSTRACT

A Goatsbeard Plant named ‘Misty Lace’ is a hybrid between Aruncus dioicus and Aruncus aethusifolius, and bears distinct habit, having perfect flowers that are borne in feathery, elongate terminal panicles.

### CROSS-REFERENCE TO RELATED APPLICATIONS

None applicable

### STATEMENT AS TO RIGHTS TO INVENTIONS MADE UNDER FEDERALLY-SPONSORED RESEARCH AND DEVELOPMENT

Not applicable

### BACKGROUND OF THE INVENTION

Aruncus dioicus×Aruncus aethusifolius Hybrid named ‘Misty Lace’

1. Field of the Invention

The present invention comprises a new and distinct plant of Aruncus, which has been given the name ‘Misty Lace’. The plant is a hybrid between Aruncus dioicus and Aruncus aethusifolius, and bears distinct habit and floral characteristics which differentiate it from existing cultivars. The plants were natural hybrids which occurred in 1999 in Monkton, Md., in the garden of Richard Simon. Three plants were taken back to the University of Georgia, Athens, Ga. for subsequent evaluation under Southeastern environmental conditions. Of the three evaluated for heat tolerance, only one plant remained alive and thrived. This single plant was the basis of this new cultivar. The plant will be used primarily as an ornamental perennial. Asexual reproduction of Aruncus ‘Misty Lace’ has been successfully accomplished through tissue culture protocol, developed by Dr. Wetzstein at the University of Georgia. This is the only means known to the inventors to rapidly reproduce this cultivar for commercial introduction. The novel habit of the plant has been consistently produced in plants derived from tissue culture.

2. Description of Relevant Prior Art

Goatsbeard, the common name for the genus, Aruncus, is a well-known genus in ornamental horticulture. Presently, the most common species available commercially is the 1.2-1.8 m common goatsbeard, A. dioicus, followed by the dwarf 0.2-0.3 m tall Korean goatsbeard, A. aethusifolius. The invention is a hybrid between the two, with a plant height of 0.3-0.5 m. ‘Misty Lace’ is distinguished from all other varieties of Aruncus known to the inventors by its plant and leaf size.

### SUMMARY OF THE INVENTION

Aruncus ‘Misty Lace’ is a hybrid between A. dioicus and A. aethusifolius, with intermediate plant and leaf size.

### BRIEF DESCRIPTION OF DRAWINGS

The accompanying illustration shows characteristics of the new cultivar in a photograph as true to color as is reasonably possible to make in illustrations of this nature.

FIG. 1 shows the habit and flowers of a typical 1 year old specimen growing in the University of Georgia Trial Gardens in Athens, Ga.

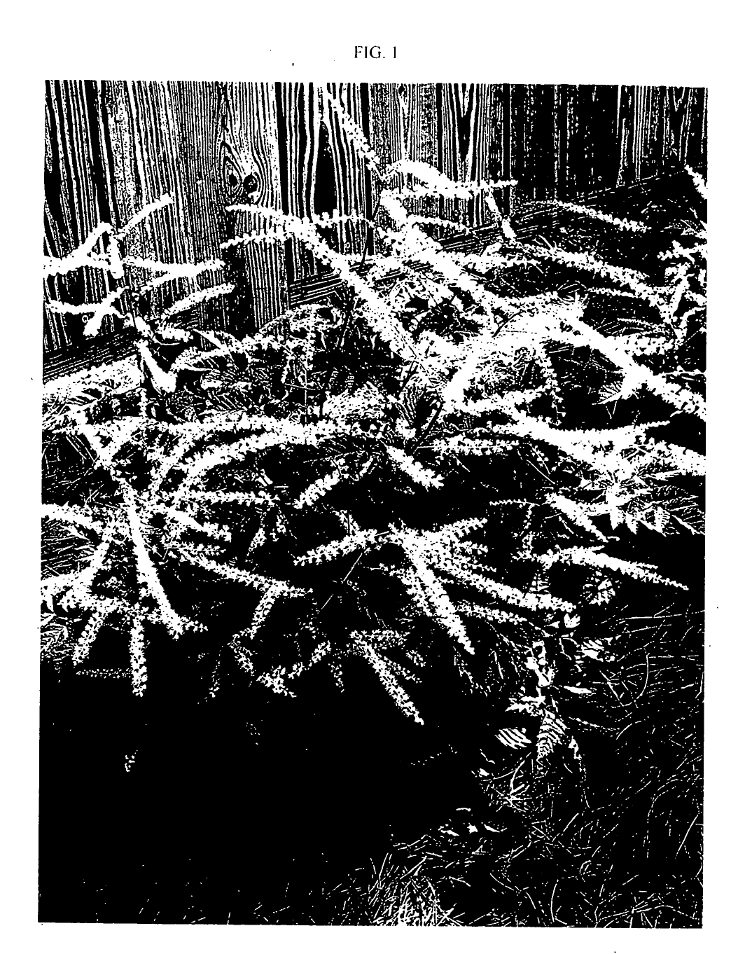

### BOTANICAL DESCRIPTION OF THE PLANT

This plant is a rhizomatous herbaceous perennial species that generally grows 30-50 cm tall at maturity in Georgia. It is a hybrid between the 2 species, A. aethusifolius and A. dioicus and is distinguished by having an intermediate habit between the two, although it favors A. aethusifolius in height. The following description defines the overall appearance of one year old plants as grown under normal conditions in The University of Georgia Horticultural Trial Gardens in Athens, Ga., U.S.A. (USDA Zone 7b)

The summers are hot and humid ranging from 75-100 F (24-38 C) the average being −85 F (29 C), and winters with average day temperatures of approximately 40 F (4 C) Colors are based on The Royal Horticultural Color Chart (1995)
Plant: Size 30-50 cm high with similar diameter:
Stems: The plants have rounded stems, about 25.4 cm in length, above which is the terminal peduncle, the flower stem. The internode length averages from 3.8-5.1 cm and the color is RHS 178A, during the early spring, and RHS 137A during the summer months.

Leaf: Leaves occur basally and on the stem and are approximately 12-16 cm long and up to 15 cm wide at their furthest point. They are doubly serrate in margin with an acuminate apex and a glabrous upper and lower surface. The leaflets themselves range from 2-4 cm in length and 2-3 cm in width. The colors of the upper and lower surfaces of the leaves are RHS 137 A and RHS 137C, respectively. Leaves are arranged alternately and are bi- to tri-pinnately compound with one ovate terminal leaflet and 3-4 pairs of pinnately arranged side leaflets. The leaves are petiolate with the petiole approximately 6 cm long with the terminal leaflet and the first side pair being sessile. The remaining 2-3 leaflet pairs are petiolate with the petiole being 0.5 cm on the second side pair and 1-2 cm on the remaining 1-2 side pairs. Table 1 presents a comparison of leaf and plant measurements and flower color among ‘Misty Lace’ and its parent species, A. aethusifolius and A. dioicus.

TABLE 1
Average leaf size, plant height and flower
color for selected Aruncus species

| | | | Height of | Color of |
|---|---|---|---|---|
| | Leaf Length | Leaf Width | Mature Plant | Flower |
| | (cm) | (cm) | (cm) | RHS |
| A. ‘Misty Lace’ | 16 | 15 | 30-50 | 155A |
| A. aethusifolius | 10 | 9 | 20-40 | 155C |
| A. dioicus | 90 | 75 | 122-182 | 155D |

Inflorescence: Flowers are perfect and are borne in feathery, elongate terminal panicles. All flowers in the inflorescence are similar in size and color, with the peduncle averaging 2-4 cm and 1 cm diameter. The color of the peduncle is RHS 137A. The mature terminal inflorescences are approximately 18 cm in length and contain approximately 250 flowers per panicle, each measuring less than 1.3 cm in diameter. The axillary inflorescences are approximately 10 cm in length and consist of approximately 35 flowers. The corolla contains 5 petals and is spatulate in shape. Flowers have 20-30 stamens.
   Timing.—Flowers are produced in mid and late spring, May-June in Athens, Ga. Plants, which were grown in 4 inch (10 cm) containers, were placed in the Horticulture Trial Garden at the University of Georgia in the spring of 2001. After one winter, plants averaged four stems with three inflorescences each. At the end of the second and subsequent winters, plants averaged 8 stems with 4-6 inflorescences each.
   Color.—The corolla of the flower is RHS 155A. The stamens of the flower are RHS 12C.
   Fragrance.—None.
   Seed.—Seed has been observed, but viability unknown.
Other characteristics: The plant has been observed for four years in Athens, Ga. and has thrived in the heat and humidity in the garden. In general, Aruncus do poorly under such conditions, but this plant has been perfectly adaptable to at least USDA zone 7b conditions. Also, in 2002-2003, plants were trailed at the University of Georgia Blairsville Research Station, the University of Florida, Gainesville, Fla., the JC Raulston Arboretum, Raleigh, N.C., the Chicago Botanic Garden, the University of Maine, Orono, Me., and the Ulbrich Botanic Garden, Madison Wis. In all cases except Gainesville, Fla., plants survived the winter of 2002 and flowered in the spring of 2003.

1. A new and distinct Aruncus plant, as herein shown and described.
